(19) United States
(12) Patent Application Publication
(10) Pub. No.: US 2022/0062497 A1
(43) Pub. Date: Mar. 3, 2022

(54) Lyophilized Cured Polymeric Foam Plug

(71) Applicant: Ethicon, Inc., Somerville, NJ (US)

(72) Inventors: Salim Ghodbane, Piscataway, NJ (US); Ashley Deanglis, Skillman, NJ (US); Sridevi Dhanaraj, Raritan, NJ (US)

(21) Appl. No.: 17/005,075

(22) Filed: Aug. 27, 2020

(51) Int. Cl. A61L 26/00; A61B 10/02; A61B 17/00

(52) U.S. Cl. CPC A61L 26/0071; A61L 26/0052; A61L 26/0033; A61L 26/0066; A61L 26/0019; A61M 25/0102; A61B 17/0057; A61L 2430/34; A61L 2400/06; A61L 2300/442; A61B 10/0233

(57) ABSTRACT

The present invention is directed to dry lyophilized foam plugs that are a polymeric reaction product of at least a pair of co-reactive polyethylene glycol having reactive moieties in which substantially all of reactive moieties have reacted prior to lyophilization or blend of biomaterial and a reactive polyethylene glycol or plasma derived biomaterial reaction product and wherein the plug has an overall pore void content of about 30-45%, and a microporous structure with an average pore generally between 20 and 95 μm.

The present invention is directed to a lyophilized, cured polymeric hydrogel foam plug that is particularly suited for lung tract sealing.

### BACKGROUND

Hydrogels, meaning polymeric materials that absorb and swell in the presence of an aqueous solution, are known in various forms. The ability to absorb, swell and yet not dissolve is due to physical or chemical crosslinkage of the hydrophilic polymer chains. Hydrogels can be prepared starting from monomers, prepolymers or existing hydrophilic polymers.

Percutaneous transthoracic needle biopsy procedures are known. Image-guided percutaneous transthoracic needle biopsy (PTNB) is an established procedure for patients with suspected pathologic conditions, such as bronchogenic carcinoma[1]. The goal of the procedure is to obtain tissue for cytologic or histologic examination. The procedure is typically performed with image guidance by a radiologist. Imaging modalities utilized include fluoroscopy, computed tomography (CT), and ultrasound. Ultrasound is the safest, quickest, and least expensive method; however, it is only useful with very superficial samples [2]. When lesions are not suitable for ultrasound, CT is the preferred imaging modality[2].

PTNB is classified according to the type of needle. Fine needle aspiration biopsy is performed to provide cytological specimens and larger diameter cutting needles produce histological specimens[2]. Historically, cutting needles have been associated with a relatively high incidence of complications but, with the introduction of automated cutting needles, recent studies have demonstrated comparable rates between fine needle aspiration and cutting needles[2].

During PTNB, an aspiration (18-22 gauge) or cutting needle (14-20 gauge) is placed under image guidance for sample recovery[1]. A coaxial technique may be used to allow for multiple passes within the lung tract and to reduce the number of pleural punctures[3]. In this technique, a thin walled introducer needle (13-19 gauge) is first inserted, localized to the lesion, and subsequently the aspiration or cutting needle is inserted[1].

Although the procedure is considered safe and effective, the incidence of pneumothorax is still significant with ranges from 12 to 61%, with 2 to 15% requiring a chest drain[2, 4]. The risk of pneumothorax increases significantly if the lesion is not adjacent to the pleura[5]. Most complications occur immediately or within the first hour following the biopsy. Therefore, following the procedure, the patient is placed in a puncture-site-down position and remain under supervision for at least 1 hour[1, 2]. The patient may present shortness of breath, chest pain, and hypoxia[6]. Most acutely symptomatic pneumothoraxes are detectable via chest radiograph. Patients observed with pneumothoraxes are administered oxygen to speed resolution of pneumothoraxes[1].

Transbronchial needle aspiration (TBNA) is a minimally invasive technique allowing for the sampling of mediastinal nodes. When integrated with endobronchial ultrasonography (EBUS), accurate definition of mediastinal structures is possible[7]. Modern devices integrate an ultrasonic bronchoscope into the needle allowing for real time visualization of the area of interest. The diagnostic yield of EBUS-TBNA in lung cancer screening has been reported with a sensitivity as high as 95.7%[8]. As a result, EBUS-TBNA is becoming widely adopted as the standard of care for sampling mediastinal lymph nodes[9].

EBUS devices consist of a transducer and a processor. The transducer produces and receives sound waves. The processor integrates the reflected sound, generating images. The probe includes a balloon which can be inflated to improve contact with the airways. EBUS-TBNA devices include an ultrasound linear processing array and a retractable needle[10]. EBUS-TBNA was originally performed with a dedicated 22-gauge aspiration needle; however, larger 21-gauge needles were introduced more recently[11]. EBUS-TBNA are carried out in the proximal lumen of level 9 bronchi, as they are restricted by the outer diameter of the bronchoscope (6.9 mm) [10, 12]. Although complications are very low in EBUS-TBNA, incidence of pneumothorax is still significant. The rate of pneumothorax has been estimated from 0.53% to 16.7% following EBUS-TBNA [9, 12].

In most institutions worldwide, the choice between TBNA or PTNB still lacks a standardized strategy[13]. The choice is typically influenced by environmental factors such as operator experience or institution resources. There is not an established algorithm based on clinical scenarios. However, PTNB is typically preferred for lesions near the visceral pleura and TBNA is preferred for those near the airways.

Patients in which enlarging pneumothoraxes are observed must be treated with the placement of a chest tube[1]. However, there is no universally accepted approach to reduce pneumothorax rate[14]. Multiple solutions have been employed to reduce the incidence of pneumothoraxes. Several authors have investigated techniques, including the rapid roll over[14] and deep expiration and breath-hold technique[15], to reduce the rate of pneumothorax but these techniques have only shown mild/moderate effects, with a risk reduction of 0.1-15.7%[15].

Therefore, others have investigated the instillation of various sealant materials into the tract, including autologous blood clot[16], fibrin glue[17], and gelatinous foam[18, 19], but none have achieved widespread use in daily practice[19]. These methods have also suffered from variable results, possibly a result of operator-dependence and variations in practice[20]. Autologous blood clot has demonstrated moderate efficacy but suffers from the long preparation times in the operating room. Although fibrin glue and gelatin techniques have demonstrated some promising published data, they have not been studied extensively.

More recently, a synthetic polyethylene glycol plug has been developed as part of the BioSentry Tract Sealant System (Angiodynamics) [20-22]. In a randomized, multicenter clinical trial, BioSentry resulted in the absence of pneumothorax in 85% of patients which was statistically greater than the control group (69%)[21]. However, the solid nature of the plug induces a foreign body giant cell reaction and an encapsulation of the hydrogel by 21 days [23].

### SUMMARY OF THE INVENTION

The present invention is directed to dry lyophilized foam plugs that are a polymeric reaction product of at least a pair of co-reactive polyethylene glycol having reactive moieties in which substantially all of reactive moieties have reacted prior to lyophilization and wherein the plug has an overall pore void content of about 30-45%, and a microporous structure with an average pore generally between 20 and 95 μm. The average pore size is determined using micro-CT analysis in which the average represents a value wherein at least about 80% of the volume of pores that are within the range.

The present invention is also directed to dry lyophilized foam plugs that are a polymeric reaction product of at least one biomaterial available electrophilic reactive moieties and at least one reactive polyethylene glycol having nucleophilic reactive moieties in which substantially all of reactive moieties have reacted prior to lyophilization and wherein the plug has an overall pore void content of about 30-45%, and a microporous structure with an average pore generally between 20 and 95 μm.

The present invention is also directed to dry lyophilized foam fibrin plugs that are a polymeric reaction product of a self-reactive derivative of fibrinogen and an activator component that generates self-reactive fibrin(ogen) derivatives in which substantially all of fibrinogen have been activated to form a fibrin plug prior to lyophilization and wherein the fibrin plug has an overall pore void content of about 30-45%, and a microporous structure with an average pore generally between 20 and 95 μm.

The present invention is also directed to methods of sealing lung or bronchial tissue having one or more tracts by inserting or otherwise delivering a foam plug according to any of the aforementioned examples into a defect. In another embodiment, the method further includes the step of applying a liquid sealant in proximity to the plugs described herein. In another embodiment, the plug can be applied by passing through a coaxial needle following a needle biopsy treatment. In another embodiment, the plug can have a diameter of 10-20 mm and is applied using an applicator that is equal to or less than the diameter of a lung tract resulting from the removal of tumorous tissue. In another embodiment, the plugs described herein can pass through a coaxial needle (0.69 to 1.8 mm) using a stylet.

The foam plugs described herein can have one or more perforations introduced using mechanical means, such punch, that are greater than 100 μm. The foam plug described herein can have one or more molded or cut perforations greater than 200 μm. In one embodiment, a post-biopsy plug as described herein can have a diameter prior to application of about 0.4 to 2 mm. In one embodiment, a post-tumor removal plug as described herein can have a diameter prior to application of about 10 to 20 mm. In one embodiment, a plug as described herein can further include a contrast agent and/or a therapeutic agent.

In one embodiment, a plug as described herein is the reaction product of synthetic polymeric components (4 Arm PEG-Amine (5k) and 4 Arm PEG-SG (20k)). In one embodiment, a plug as described herein can be a solid foamed structure that is stabilized with a surfactant.

In one embodiment, the present invention is a fibrin plug wherein foamed structure comprises sufficient factor XIII to enhance the mechanical integrity and stability.

In one embodiment, a plug as described herein can have one or more ribbed sections, one or more barbs, and/or one or more regions with undulating topography. The ribbed section, barb or undulating region can be molded and/or cut or shaped after lyophilization.

### DETAILED DESCRIPTION

The present invention relates to a pre-formed, polymerized lyophilized biosynthetic, synthetic, or biologic foam plug or perforated foam plugs with a preferably cylindrical geometry that can be applied into lung tracts, following tumor removal and/or biopsy of tumor nodules through a percutaneous or endobronchial approach. The plug can be utilized for large lung tract (up to a 20 mm diameter) or needle biopsy tract sealing (0.41-1.8 mm or 13-22 needle gauge). The large plugs can be applied using an applicator that is equal to or less than the diameter of the lung tract. The applicator can be inserted into the lung tract and retracted as the plug is inserted. For percutaneous approach, the needle tract plugs can be inserted via a coaxial needle. The plug can be passed through the coaxial needle using a stylet. For the endobronchial approach, an appropriate endobronchial catheter system may be used. The foam plug can expand immediately upon hydration causing a mechanical seal to form.

One embodiment of the device is a foam plug consisting of a biosynthetic combination of a proteinaceous component, such as albumin, and polyethylene glycol-succinimidyl glutarate (PEG-SG) component having an air/gas content of about 30-45% by volume that has fully crosslinked, quickly frozen at negative 80° C. and then subjected to lyophilization. The albumin component can be natural, such as human serum albumin, or recombinantly produced. The albumin is provided in the foam forming mixture at a concentration of from about 100-300 mg/ml, preferably about 100 mg/ml. The PEG-SG component can be 2, 3, 4, 6, 8, etc. arm polyethylene glycol succinimidyl glutarate, more preferably a 4-arm polyethylene glycol succinimidyl glutarate (PEG-SG4) having a molecular weight from about 1000 to 20,000 Daltons added to the foam forming mixture at a concentration of about 50 mg/ml.

There are three potential sources of porosity in lyophilized foam plugs: 1) porosity of the polymer structure (100-1000 Angstrom); 2) the porosity introduced by the lyophilization process (20-40 μm); and 3) porosity caused by foaming (20-270 μm). In this particular system, the pores introduced by foaming are believed to produce an acceleration in hydration rate . The resulting plug is elastic, deformable, compressible and flexible (radially and axially) prior to wetting and/or application.

Fully crosslinked or fully reacted does not mean that every SG group has reacted with the available nucleophile. It is possible that some SG groups may hydrolyze prior to crosslinking or during crosslinking. Additionally, some SG and/or nucleophilic group are not available for reaction due to steric hindrance. Under the intent is that under the selected reaction conditions, which are defined by the type and amounts of electrophile and nucleophile and pH of the reaction, all available groups have reacted with their corresponding reactant.

The step of quickly freezing, in about one hour or less, the mixture at −80 C is advantageous because the resulting product after the freezing step has a homogenous distribution of ice crystals which impacts reproducible characteristics.

The preferred lyophilization cycle conditions were maintained in a freeze dryer at a condenser temperature of −70° C. with the following cycle times:

| Shelf Temperature | Pressure | Time |
|---|---|---|
| (° C.) | (mTorr) | (min) |
| −40 | 20 | 240 |
| −30 | 20 | 240 |
| −20 | 20 | 300 |
| −10 | 20 | 240 |
| 0 | 20 | 180 |
| 10 | 20 | 120 |
| 20 | 20 | 30 |

A second embodiment of the device, the foam plug consists of a reaction product of only synthetic polymeric components, such as a multi-arm polyethylene glycol PEG-Amine and a multi-arm PEG-SG to form a PEG-based foam having about 30-45% air/gas content, wherein the foam structure is stabilized by addition of a surfactant, such as Polysorbate-20 that has substantially fully reacted available moieties, frozen at −80° C., and lyophilized with a diameter of from about 0.69-1.8 mm. The resulting plug is tough, elastic, deformable, and flexible.

The PEG-Amine component consists of poly(ethylene glycol) amine macromers, such as linear bifunctional poly(ethylene glycol) amine or n-arm poly(ethylene glycol) amines, where n is an integer of 2 more. A preferred PEG-Amine for this embodiment has four arms and a molecular weight of at least 5000 Daltons (5k).

The PEG-SG component consists of poly(ethylene glycol) SG macromers, such as linear bifunctional poly(ethylene glycol) SG or n-arm poly(ethylene glycol) SG, where n is an integer of 2 or more. A preferred PEG-SG for this embodiment has four arms and a molecular weight of at least 5000 Daltons (5k).

A third embodiment of the device could be a plasma derived biologics foam. Such a plasma derived biologic foam could be created by combining a fibrinogen component with an activator, such as thrombin, at a low activity (2-50 IU/mL) to avoid rapid polymerization and introducing air/gas at the desired level of about 30-45% air/gas content. Once the available fibrin polymerizes, the resulting biologic foam is frozen at −80° C. and lyophilized.

Each of the foam plugs described above are packaged, sterilized and applied for use in a dry state meaning that the plugs do not contain significant moisture other than the result of the ambient surroundings. More preferably, the plugs are considered dry as having a moisture content under ordinary room conditions of less than 8%, more preferably less than about 5%, most preferably less than 3%.

Each of the foam plugs described above are reaction products of plasma derived components, synthetic reactive polymers and/or biomaterials having polymerizable reactive groups. A reaction product, for purposes of this application, refers to a material has been subjected to appropriate time and conditions to cause all or substantially all available reactive groups to react with the co-reactive moieties and/or with chemical crosslinking agents, preferably having at least two reactive groups.

The foam plugs as described above can be applied in combination with a biosynthetic, synthetic, or biological liquid sealant in order to achieve pneumostasis and hemostasis control. The liquid sealants used in combination with the plug can include a liquid solution of nucleophile (example: albumin or PEG- Amine) and PEG-SG that is pre-mixed immediately prior to use or a biological liquid sealant (i.e. a fibrin sealant formulation containing a fibrinogen component and a fibrinogen activator or polymerizing agent). These liquid sealants can be applied before, during, or after insertion of the foam plug. The liquid sealants should be allowed to crosslink for a sufficient period of time, from 5 seconds to 5 minutes, to form a seal within and at the surface of the lung preventing air leaks.

A particularly preferred foam can be formed by reacting 25-100 mg/mL of a multi-arm PEG having more than three (3) electrophilic groups with a molecular weight of 5 kDa to 20 kDa, with about 50-200 mg/mL albumin, and about 30-45% air content. A particularly preferred reaction formulation comprises 75 mg/mL of the 4 Arm PEG-SG (20k), 10% concentration albumin (meaning 100 mg/mL), in general, % refers to number of grams of material in 100 mL water, 50 mM carbonate buffer (pH=9.0).

The preferred foam plugs exhibit fast hydration time as a result of a porous structure of the foam that allows for faster penetration of the plug by water leading to faster swelling of the plug for increased mechanical sealing. The volumetric increase can be controlled based on the original foam (container) dimensions.

The preferred foam plugs also exhibit fast absorption time because the foam is comprised of a large percentage of air (or gas) so that the mass of material implanted is reduced allowing for faster absorption during healing. The absorption time of the PEG foam is further tunable based on the bio-degradability of the PEG cross-linker. The absorption time of a plasma biologic foam can be modulated by varying the amount of fibrinogen and Factor XIII in the biologic foam.

The preferred foam plugs are compliant, compressible foam that allows for insertion into a lung tract and subsequent expansion for an immediate mechanical seal. The shape of the foam can be cylindrical or tapered to create differential pressure on the tissue within the tract.

The preferred foam plugs have sufficient flexibility once hydrated so that the foam can expand and contract with the natural movement of a breathing lung.

The preferred foam plugs have high volume to mass ratio because most of the foam contains air (or gas) with the liquid phase representing only 55-70% of the volume, i.e., 30-45% of the volume of the foam plug is gas.

The preferred foam plugs can optionally include a contrast agent, such as Iohexol, also known as Omnipaque and Hexopaque, which is a contrast agent used during X-ray imaging. The latter contrast agent has been shown to not impact the quality of the PEG-albumin foam.

The invention describes a lyophilized biosynthetic, synthetic or biologic foam plug preferably cylindrical to be applied into lung tracts following a percutaneous or endobronchial biopsy procedure to prevent pneumothorax. Percutaneous approaches can include either a needle biopsy procedure (0.41 to 1.8 mm diameter) or a coring procedure to remove a tumor nodule (<20 mm diameter). The foam plug can expand immediately upon hydration causing a mechanical seal to form. The foam plug may be applied in combination with a biosynthetic, synthetic, or biological liquid sealant to achieve pneumatosis and hemostasis control.

One embodiment of the device is a foam plug or perforated foam plugs consisting of a biosynthetic combination of a proteinaceous component (example: albumin) and polyethylene glycol-succinimidyl glutarate component (PEG-SG) (30-45% air/gas content) that has fully crosslinked, frozen at −80° C., and lyophilized with a diameter of 0.41 to 1.8 mm. The resulting plug is tough, elastic, deformable, and flexible. The plug can be passed through the coaxial needle following a needle biopsy procedure.

In another embodiment of the device, the biosynthetic plug can possess a diameter of 10-20 mm and be applied using an applicator that is equal to or less than the diameter of a lung tract resulting from the removal of tumorous tissue (<20 mm). The applicator can be inserted into the lung tract and retracted as the plug is inserted.

In another embodiment of the device, the biodegradable material can be combined with non-ionic contrast agents (example: Iohexol) for radiopacity. The contrast agents can allow for localization of the site at a later date.

In another embodiment of the device, the biodegradable material can be combined with contrast agents to facilitate detection by magnetic resonance imaging. The contrast agents can allow for localization of the site at a later date.

In another embodiment of the device, the biodegradable material can be combined with radioactive agents for radiation detection methods. The radioactive agents can allow for localization of the site at a later date.

In another embodiment of the device, the biodegradable material can be combined with therapeutic agents. The therapeutic agents (e.g. chemotherapeutic agents) can provide localized drug delivery for management of the cancer.

In a second embodiment of the device, the foam is a reaction product of only synthetic polymeric components (example: 4 Arm PEG-Amine (5k) and 4 Arm PEG-SG (20k)), to form a PEG-based foam having an air/gas content of about 30-45% by volume, and foam structure stabilized with a surfactant (example: Polysorbate-20) that has reacted substantially all available reactive moieties, frozen at −80° C., and lyophilized with a diameter of 0.41-1.8 mm. The resulting plug is tough, elastic, deformable, and flexible. The plug can be passed through the coaxial needle (0.69 to 1.8 mm) using a stylet.

In another embodiment of the device, the synthetic plug can possess a diameter of 10-20 mm and be applied using an applicator that is equal to or less than the diameter of a lung tract resulting from the removal of tumorous tissue (<20 mm). The applicator can be inserted into the lung tract and retracted as the plug is inserted.

In a third embodiment of the device, the foam can be composed of fibrin obtained by combining fibrinogen with a fibrinogen activator or polymerizing agent, such as thrombin at low activity and foaming the mixture by introducing air or gas. The aerated fibrin is allowed to polymerize, frozen at −80° C., and lyophilized with a diameter of 0.41-1.8 mm. The resulting plug is tough, elastic, deformable, and flexible. The plug can be passed through the coaxial needle (0.69 to 1.8 mm) using a stylet.

In another embodiment of the device, the biological plug can possess a diameter of <20 mm and be applied using an applicator that is equal to or less than the diameter of a lung tract resulting from the removal of tumorous tissue (approximately 20 mm). The applicator can be inserted into the lung tract and retracted as the plug is inserted.

In another embodiment of the device, the fibrin foam can be crosslinked using factor XIII to enhance the mechanical integrity and stability of the foam. In another embodiment of the device, the plug can be fabricated to include ribbed features to improve the ability of the plug to resist extrusion under pressure.

In another embodiment of the device, the plug can possess oversized dimensions to improve the ability of the plug to resist extrusion under pressure.

In another embodiment of the device, the plug can possess additional pores to improve the hydration and resorption of the plugs fabricated via perforations, molds with removable pins, etc.

In another embodiment of the device, the plug is inserted using a stylet. In another embodiment of the device, the plug is inserted using pneumatic pressure until in the desired location. In another embodiment of the device, the plug is held with a cylindrical mesh, moved into place, and the plug is deployed by expanding the mesh.

### EXAMPLES

### Example 1

### Biosynthetic, Synthetic, and Biological Plug Formulations

1. Biosynthetic Liquid: 75 mg/mL 4 Arm PEG-SG-20k, 10% albumin, 50 mM carbonate (pH=9.0)
2. Biosynthetic High-Density Foam: 75 mg/mL 4 Arm PEG-SG-20k, 10% albumin, 50 mM carbonate (pH=9.0) (2:1 liquid to air ratio, 66% Air)
3. Biosynthetic Low-Density Foam: 75 mg/mL 4 Arm PEG-SG-20k, 10% albumin, 50 mM carbonate (pH=9.0) (1:2 liquid to air ratio, 33% Air)
4. Synthetic Liquid: 75 mg/mL 4 Arm PEG-SG-20k, 57 mg/mL 4 Arm PEG-NH2-5k, 50 mM carbonate (pH=9.0)
5. Fibrin Sealant

These formulations were hydrated in phosphate buffered saline. The increase in mass due to hydration was measured at 5 and 10 minutes. The synthetic formulation hydrated 5-fold more than the biosynthetic and fibrin sealant formulations. There was no significant difference in hydration between the biosynthetic and fibrin sealant formulations. Although approximately 82% of the hydration occurred by 5 minutes, there was a significant difference in hydration between 5 and 10 minutes. Although the synthetic formulation demonstrated significantly greater hydration than the other formulations, the synthetic formulation possesses poor cohesive properties. This is demonstrated by a significantly lower tensile stiffness and tensile ultimate stress. This reduction in cohesive properties will lead to poorer sealing in a needle tract.

### Example 2

A biosynthetic formulation of 75 mg/mL 4 Arm PEG-SG-20k, 10% albumin, 50 mM carbonate (pH=9.0) was tested. For each group tested, 750 mg PEG-SG4-20k was dissolved in 5 mL, 100 mM carbonate buffer (pH=9.0). The PEG-SG4-20k was loaded into a 20 mL slip tip syringe and the plunger was positioned to the graduation corresponding to the table below. 5 mL 20% albumin was loaded into a 20 mL syringe and the plunger was positioned to the graduation corresponding to the table below. The syringes were connected using a dual syringe connector and the solutions were passed 20 times. The syringe was immediately connected to a needle and the foam was expressed slowly until several drops were assessed. The needle tip was then impaled on a rubber stopper and the needle was removed from the syringe. Once crosslinked completely, the needle was removed from the rubber stopper.

| Air | Liquid | Air | Syringe Graduation |
|---|---|---|---|
| Content | (mL) | (mL) | (mL) |
| 0% | 2.5 | 0.00 | 2.50 |
| 15% | 2.5 | 0.44 | 2.94 |
| 30% | 2.5 | 1.07 | 3.57 |
| 45% | 2.5 | 2.05 | 4.55 |
| 60% | 2.5 | 3.75 | 6.25 |

Foaming of formulations increased the hydration rate of the biosynthetic formulation. The effect of foaming was assessed within the biosynthetic formulation with 0% (solid), 33%, and 66% air content. The foamed plugs (33% and 66% air) hydrated to approximately 2.5-fold more than the solid plug. The foamed plugs were significantly more hydrated than the solid plug, although there was no significant difference between the 33% and 66% air plugs.

Air content of 0, 15, 30, 45 and 60% were tested. Air content in lyophilized foams had a significant effect (p<0.01, one-way ANOVA) on hydration at 5 minutes. Specifically, 45% air content demonstrated significantly greater hydration than 0 and 15% air foams. In addition, a test for equal variance demonstrated that the variances in all groups were significantly different (p<0.01). Therefore, a series of tests for two variances was performed that showed the variance of the 60% air content foam was significantly greater than the 30% and 45% air content foams, demonstrating the inconsistent hydration rate of the foam with 60% air.

Air content in lyophilized foams had a significant effect (p<0.01, one-way ANOVA) on hydration at 10 minutes. Specifically, foams with greater than or equal to 30% air content demonstrated significantly greater hydration than 0 and 15% air foams. The variances of the 30%, 45%, and 60% foams were not significantly different at 10 minutes. Therefore, a preformed, lyophilized foam plug of 30% to 45% air content results in best hydration with the least variance at 5 minutes. A foam of 30% to 60% air content results in best hydration at 10 minutes.

### Example 3

### Ex-Vivo Results

Foam plug prototypes were evaluated for their ability to achieve pneumatosis in an ex vivo porcine lung model. In this model, lung plucks were harvested fresh on the day of testing and kept moist until testing. Prior to testing, the lungs were placed on a ventilator to recruit collapsed alveoli (goal is to open up collapsed airless alveoli). At the time of testing, lungs were connected to a Respironics respirator to precisely control the pressure during ventilation cycles. The pressure was set to an inspiration pressure of 25 cm water and expiration pressure of 5 cm water (20 cm water differential).

Lung puncture defects were created with a 12 mm punch (Acu-punch) on lungs that were connected to the respirator and cycling. The defect size was measured after the punch to be approximately 1.5 cm diameter and 3 cm deep on inflated lung. The air leak in the defect was assessed as severe with a bubble test. When prototypes were applied, pressure was reduced to inspiration pressure of 10 cm water and expiration pressure of 10 cm water (no change) to keep the lungs expanded.

After prototype application, typically topical compression was placed on prototype for 1 min while the lung was still expanded and under positive pressure. To test performance, lung was ventilated starting at low pressure and increasing to inspiration pressure of 25 cm water and expiration pressure of 5 cm water (20 cm water differential). Bubble test was performed by passing saline over puncture site and recording for presence and severity of air leak. For an additional challenge, ventilation pressures were increased to inspiration pressure of 40 cm water and expiration pressure of 5 cm water (35 cm water differential). After pressure testing, prototypes were pulled from puncture site and adherence to surrounding tissue was qualitatively assessed. The specific prototypes which were tested are listed in the caption for each image. Both foam plug prototypes sealed the air leaks at both 20 and 35 cm water when used in combination with a fibrin sealant, Evicel or PEG-albumin liquid sealant.

### Example 4

Needle tract sealing prototypes were assessed in an ex vivo porcine lung model. The goal of the testing was to evaluate pneumostasis effectiveness of pre-formed plug/paste sealant prototypes to close a pleural and parenchymal lesion in the lung after a percutaneous or thoracoscopic needle lung biopsy. Lung plucks were freshly harvested on the day of testing. Immediately, prior to testing, the lungs were placed on a ventilator to recruit collapsed alveoli. The lungs were connected to a Respironics respirator to precisely control the pressure during ventilation cycles. The pressure was set to an inspiration pressure of 25 cm water and expiration pressure of 5 cm water (20 cm water differential) to acclimate lungs.

During needle biopsies, the lungs were expanded by setting the respirator to a constant pressure of 10 cm water (inspiration and expiration pressure of 10 cm water). The needle tracts were created in the lungs using a 19-gauge biopsy needle that was inserted through a coaxial needle port which was positioned 3 cm deep. Prototype plugs were either inserted into the needle tract using a commercially available plug assembly and stylet or were inserted manually by pushing the plug into position using the stylet.

After prototype application/insertion, a time duration of at least 3 min was allowed for the prototype to expand and/or polymerize within the lung while under positive pressure (10 cm water). To test sealing performance, lung was ventilated at 20 cm water pressure differential (25 cm water inspiration pressure and 5 cm water expiration pressure, i.e., 20 cm water differential). A bubble test with saline was performed to assess the presence and severity of any air leak. The results obtained for specific prototypes are shown below.

### Results:

Prototype: Lyophilized Surgifoam/Surgiflo PEG Liquid Plug (L1-6). Minor leak observed at 20 cm water pressure. Leak was significantly reduced relative to untreated needle tract defect.

Prototype: Lyophilized Evicel Fibrin Sealant Plug (L1-7). Minor leak observed at 20 cm water pressure. Leak was significantly reduced relative to untreated needle tract defect.

Prototype: Lyophilized Biosynthetic liquid (PEG-SG4+Albumin) Plug (L2-1). No leaks observed at 20 cm water pressure.

Prototype: Lyophilized Biosynthetic Foam Plug (2:1 Liquid to air) (L2-5). Minor leak observed at peak pressure when ventilated at 20 cm water pressure. Leak was significantly reduced relative to untreated needle tract defect.

PCT
PCT 1. A dry lyophilized foam plug that is a polymeric reaction product of at least a pair of co-reactive polyethylene glycols having reactive moieties in which substantially all of reactive moieties have reacted prior to lyophilization and wherein the plug has an overall pore void content of about 30-45%, and a microporous structure with an average pore generally between 20 and 95 μm.
PCT 2. A dry lyophilized foam plug that is a polymeric reaction product of at least one biomaterial available electrophilic reactive moieties and at least one reactive polyethylene glycol having nucleophilic reactive moieties in which substantially all of reactive moieties have reacted prior to lyophilization and wherein the plug has an overall pore void content of about 30-45%, and a microporous structure with an average pore generally between 20 and 95 μm.
PCT 3. A dry lyophilized foam fibrin plug that is a polymeric reaction product of a self-reactive derivative of fibrinogen and an activator component that generates self-reactive fibrin(ogen) derivatives in which substantially all of reactive groups of the fibrinogen derivative have reacted to form a fibrin plug prior to lyophilization and wherein the fibrin plug has an overall pore void content of about 30-45%, and a microporous structure with an average pore generally between 20 and 95 μm.
PCT 4. A method of sealing lung or bronchial tissue having one or more tracts comprising inserting a foam plug according to any of the foregoing into a defect.
PCT 5. A plug according to any one of PCT 1 to 3 having one or more perforations that greater than 40 μm.
PCT 6. A plug according to any one of PCT 1 to 3 having one or more molded or cut perforations greater than 40 μm.
PCT 7. A post-biopsy plug according to any one of PCT 1 to 3 having a diameter prior to application of about 0.4 to 2 mm.
PCT 8. A post-tumor removal plug according to any one of PCT 1 to 3 having a diameter prior to application of about 10 to 20 mm.
PCT 9. A plug according to any one of PCT 1 to 3 further comprising a contrast agent.
PCT 10. A plug according to any one of PCT 1 to 3 further comprising a therapeutic agent.
PCT 11. A plug according to any one of PCT 1 to 3 that is the reaction product of synthetic polymeric components (4 Arm PEG-Amine and 4 Arm PEG-SG).
PCT 12. A plug according to any one of PCT 1 to 3 wherein the solid foamed structure further comprises a surfactant.
PCT 13. A fibrin plug according to any one of PCT 1 to 3 wherein foamed structure comprises sufficient factor XIII to enhance the mechanical integrity and stability.
PCT 14. A plug according to any one of PCT 1 to 3 having one or more ribbed sections, one or more barbs, and/or one or more regions with undulating topography.
PCT 15. A plug according to any one of PCT 1 to 3 wherein the ribbed section, barb or undulating region is molded and/or cut or shaped after lyophilization.
PCT 16. A method further comprising applying a liquid sealant in proximity to the plug according to any one of PCT 1 to 3.
PCT 17. A method wherein the plug according to any one of PCT 1 to 3 is applied by passing through a coaxial needle following a needle biopsy treatment.
PCT 18. A method wherein the plug according to any one of PCT 1 to 3 has a diameter of 10-20 mm and is applied using an applicator that is equal to or less than the diameter of a lung tract resulting from the removal of tumorous tissue.
PCT 19. A method wherein the plug according to any one of PCT 1 to 3 passes through a coaxial needle (0.69 to 1.8 mm) using a stylet

I/We claim:

1. A dry lyophilized foam plug that is a polymeric reaction product of at least a pair of co-reactive polyethylene glycols having reactive moieties in which substantially all of reactive moieties have reacted prior to lyophilization and wherein the plug has an overall pore void content of about 30-45%, and a microporous structure with an average pore generally between 20 and 95 μm.

2. A dry lyophilized foam plug that is a polymeric reaction product of at least one biomaterial available electrophilic reactive moieties and at least one reactive polyethylene glycol having nucleophilic reactive moieties in which substantially all of reactive moieties have reacted prior to lyophilization and wherein the plug has an overall pore void content of about 30-45%, and a microporous structure with an average pore generally between 20 and 95 μm.

3. A dry lyophilized foam fibrin plug that is a polymeric reaction product of a self-reactive derivative of fibrinogen and an activator component that generates self-reactive fibrin(ogen) derivatives in which substantially all of reactive groups of the fibrinogen derivative have reacted to form a fibrin plug prior to lyophilization and wherein the fibrin plug has an overall pore void content of about 30-45%, and a microporous structure with an average pore generally between 20 and 95 μm.

4. A method of sealing lung or bronchial tissue having one or more tracts comprising inserting a foam plug according to claim 2 into a defect.

5. A plug according to claim 2 having one or more perforations that greater than 40 μm.

6. A plug according to claim 2 having one or more molded or cut perforations greater than 40 μm.

7. A post-biopsy plug according to claim 2 having a diameter prior to application of about 0.4 to 2 mm.

8. A post-tumor removal plug according to claim 2 having a diameter prior to application of about 10 to 20 mm.

9. A plug according to claim 2 further comprising a contrast agent.

10. A plug according to claim 2 further comprising a therapeutic agent.

11. A plug according to claim 1 that is the reaction product of synthetic polymeric components (4 Arm PEG-Amine and 4 Arm PEG-SG).

12. A plug according to claim 3 wherein the solid foamed structure further comprises a surfactant.

13. A fibrin plug according to claim 3 wherein foamed structure comprises sufficient factor XIII to enhance the mechanical integrity and stability.

14. A plug according to claim 2 having one or more ribbed sections, one or more barbs, and/or one or more regions with undulating topography.

15. A plug according to claim 2 wherein the ribbed section, barb or undulating region is molded and/or cut or shaped after lyophilization.

16. A method further comprising applying a liquid sealant in proximity to the plug according to claim 2.

17. A method wherein the plug according to claim 2 is applied by passing through a coaxial needle following a needle biopsy treatment.

18. A method wherein the plug according to claim 2 has a diameter of 10-20 mm and is applied using an applicator that is equal to or less than the diameter of a lung tract resulting from the removal of tumorous tissue.

19. A method wherein the plug according to claim 2 passes through a coaxial needle (0.69 to 1.8 mm) using a stylet.
